(19) United States
(12) Patent Application Publication
(10) Pub. No.: US 2018/0000616 A1
(43) Pub. Date: Jan. 4, 2018

(54) Device and Method for Treatment of Barrett's Esophagus

(71) Applicant: Peter Derk Siersema, Rotterdam (NL)

(72) Inventor: Peter Derk Siersema, Rotterdam (NL)

(21) Appl. No.: 15/600,616

(22) Filed: May 19, 2017

Related U.S. Application Data
(60) Provisional application No. 62/339,177, filed on May 20, 2016.

(51) Int. Cl. A61F 2/82; A61B 18/14; A61B 18/00; A61F 2/04

(52) U.S. Cl. CPC A61F 2/82; A61F 2002/044; A61B 18/1492; A61B 2018/00273

(57) ABSTRACT

Barrett's esophagus is a serious complication of GERD. It is characterized by the replacement of the normal stratified squamous epithelium lining of the esophagus by simple columnar epithelium with goblet cells (which are usually found lower in the gastrointestinal tract). A method comprising deploying multiple stents to cause pressure ischemia and subsequent necrosis of the mucosal layer of the esophagus which is affected with the Barrett's disease is disclosed. A pair of implantable stents having specific characteristics is disclosed which can be deployed in the esophagus to cause necrosis of the mucosal layer of the esophagus through induction pressure.

### CROSS-REFERENCE TO RELATED APPLICATIONS

The present application relies on, for priority, U.S. Patent Provisional Application No. 62/339,177, entitled “Device and Method for Treatment of Barrett's Esophagus” and filed on May 20, 2016, which is herein incorporated by reference in its entirety.

### FIELD

The present specification generally relates to the field of esophageal disease and more specifically to an implantable device and method for treating Barrett's esophagus.

### BACKGROUND

After food is swallowed, it passes through the esophagus, which is a hollow, muscular tube that runs from the mouth to the stomach. The lower esophageal sphincter, which is a ring of muscle located at the end of the esophagus at the juncture between the stomach and the esophagus, keeps stomach contents from rising up into the esophagus. With gastroesophageal reflux disease (GERD), stomach contents flow upward into the esophagus, causing reflux. People with GERD may experience symptoms such as heartburn, a sour, burning sensation in the back of the throat, chronic cough, laryngitis, and nausea. Barrett's esophagus, a serious complication of GERD, is characterized by the replacement of the normal stratified squamous epithelium lining of the esophagus by simple columnar epithelium with goblet cells (which are usually found lower in the gastrointestinal tract). It should be noted that Barrett's esophagus and GERD are not mutually inclusive, meaning that not everyone with GERD develops Barrett's esophagus and not everyone with Barrett's esophagus has GERD. Long-term GERD, however, is the primary risk factor and about 10% of people with chronic symptoms of GERD develop Barrett's esophagus. In patients with frequent acid reflux, the normal cells in the esophagus may eventually be replaced by cells that are similar to cells generally found in the tissue lining of the intestine.

Barrett's esophagus can be diagnosed while performing tests to find the cause of a patient's GERD symptoms. Physicians may diagnose Barrett's esophagus with an upper gastrointestinal (GI) endoscopy and a biopsy. For upper GI endoscopy, a physician uses an endoscope to view the inside of the upper GI tract. The physician positions the endoscope through the patient's esophagus and into the stomach and duodenum. The procedure may show changes in the lining of the esophagus. A biopsy is subsequently performed to confirm the presence of Barrett's esophagus cells.

The medical significance of Barrett's esophagus is its strong association with esophageal adenocarcinoma, which is a serious, potentially fatal cancer of the esophagus, because of which Barrett's esophagus is considered a premalignant condition. Although the risk of this cancer is higher in people with Barrett's esophagus, the disease is still rare. Less than 1% of people with Barrett's esophagus develop this particular type of cancer. Nevertheless, if one is diagnosed with Barrett's esophagus, it is important to undergo a detailed endoscopic examination of the esophagus, have follow-up endoscopies at regular intervals and often begin a course of treatment with proton-pump inhibitors (PPIs), particularly when GERD symptoms are present. While Barrett's esophagus can be found in different stages of malignancy (dysplasia), it is generally recommended to treat dysplasia in all cases. Currently, the primary treatment option is ablation therapy, such as radiofrequency ablation (RFA). Cryotherapy is also a treatment option; however, it is not as well developed as RFA therapy. Barrett's esophagus, after biopsy, is classified into four general categories: non-dysplastic, low-grade dysplasia, high-grade dysplasia, and frank carcinoma. High-grade dysplasia and early stages of adenocarcinoma can be treated by endoscopic resection and new endoscopic therapies such as RF ablation, whereas advanced stages (submucosal) are generally advised to undergo surgical treatment. Non-dysplastic low-grade dysplastic patients are generally advised to undergo annual observation with endoscopy, while RFA therapy is increasingly accepted as a therapeutic option for low-grade dysplasia.

RFA therapy has proven to be a safe and effective method for treating Barrett's esophagus. Radiofrequency energy (radio waves) is delivered via a balloon mounted on a catheter to the esophagus to remove diseased tissue while minimizing injury to healthy esophageal tissue. This is referred to as ablation, or the removal or destruction of abnormal (dysplastic) tissue. Under sedation, a device is inserted through the mouth into the esophagus and is used to deliver a controlled level of energy and power to remove a thin layer of diseased tissue. Less than one second of energy exposure removes tissue to a depth of about one millimeter. The ability to provide a controlled amount of heat to a diseased tissue ensures that RFA therapy has a lower rate of complications than other forms of ablation therapy.

While RFA therapy is reasonably effective as a first line therapy to eradicate Barrett's esophagus, about 10-20% of RFA treatments do not result in complete eradication of Barrett's esophagus as Barrett's tissue is hidden under mucosa. The risk of recurrence of Barrett's esophagus is as high as 20%. Moreover, this treatment is also expensive on a per patient basis. Therefore, there is a need for developing more effective and preferably cheaper methods and systems to treat Barrett's esophagus.

### SUMMARY

In some embodiments, the present specification discloses a method for treating Barrett's esophagus in a patient comprising: identifying an affected region in the esophagus; deploying an at least partially uncovered first stent within the esophagus wherein the first stent overlaps at least the entire affected region in the esophagus; leaving said first stent within the esophagus for a first predetermined time period; deploying an at least partially covered second stent within the esophagus after said predetermined time period such that the second stent is positioned within the first stent, thereby positioning the first stent between the second stent and the affected region in the esophagus, and extends beyond the first stent by a predefined distance; and, removing the first stent and second stent after a second predetermined time period.

Optionally, the method further comprises ablating tissues in the affected region before deploying the first stent.

Optionally, the method further comprises ablating tissues in the affected region after removing the first stent and the second stent.

Optionally, said first predetermined time period is at least seven days from the deploying of the first stent.

Optionally, said second predetermined time period ranges from ten to fourteen days from the deployment of the second stent.

Optionally, the second stent is coupled to the first stent to prevent migration within the esophagus.

Optionally, the second stent is coupled to the first stent using a magnetic field.

Optionally, the second stent is coupled to the first stent using at least one of a mechanical member and an adhesive.

Optionally, when said second stent is positioned within said first stent, a mucosal layer of the esophagus is sandwiched between the first and second stents under an amount of pressure sufficient to cause pressure ischemia and subsequent necrosis of the mucosal layer of the esophagus.

Optionally, a diameter of the first stent ranges between 20 and 32 mm and a diameter of the second stent ranges between 22 and 34 mm.

Optionally, a length of the first stent is at least 2 cm greater than at least a length of the affected region and a length of the second stent is at least 2 cm greater than a length of the first stent.

Optionally, a radial force of the first stent ranges between 60 and 90 Newton at approximately 15 mm expansion and a radial force of the second stent ranges between 90 and 120 Newton at approximately 15 mm expansion.

Optionally, an axial force of the first stent ranges between 0.1 and 0.5 Newton at approximately 15 mm expansion and an axial force of the second stent ranges between 0.1 and 0.5 Newton at approximately 15 mm expansion.

Optionally, the first stent and the second stent comprise radiopaque markers positioned on at least one of a proximal end, center portion and distal end.

Optionally, the first stent comprises a flange having a diameter ranging between 24 and 36 mm, and a length ranging from 0.5 cm to 6 cm and wherein the first stent provides a radial force ranging between 70 and 100 Newton and an axial force ranging between 0.1 and 0.5 Newton at 15 mm expansion.

Optionally, the second stent comprises a flange having a diameter ranging between 26 and 38 mm, and a length ranging from 0.5 cm to 6 cm and wherein the second stent provides a radial force ranging between 70 and 100 Newton and an axial force ranging between 0.1 and 0.5 Newton at 15 mm expansion.

Optionally, at least one of a proximal and a distal portion ranging in length from 0.5 to 4 cm of the first stent is partially covered and wherein the covered portion of the first stent is placed outside the affected region and wherein at least one of a proximal and distal portion ranging in length from 0.5 to 4 cm of the second stent is partially uncovered.

Optionally, covered portions of the first stent and second stent comprise material made of at least one of silicone, polyethylene, and polyurethane.

Optionally, the first stent is configured such that it can be released through at least one of a proximal section of the first stent and a distal section of the first stent.

Optionally, the second stent is configured such that it can be released through at least one of a proximal section of the second stent and a distal section of the second stent.

The aforementioned and other embodiments of the present shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1A illustrates an epithelium lining of a normal, healthy esophagus;

FIG. 1B illustrates an epithelium lining of an esophagus affected with Barrett's syndrome;

FIG. 2A illustrates an exemplary embodiment of a partially uncovered stent which may be deployed, in accordance with an embodiment of the present specification;

FIG. 2B illustrates an exemplary embodiment of a fully covered stent which may be deployed, in accordance with an embodiment of the present specification;

FIG. 3 is a table listing properties of stents that may be employed, in accordance with an embodiment of the present specification;

FIG. 4 is a flow chart describing a method for treating Barrett's esophagus, in accordance with an embodiment of the present specification;

FIG. 5A is a pictorial representation of a first stent deployed within a lumen of a Barrett's esophagus, in accordance with an embodiment of the present specification;

FIG. 5B is a pictorial representation of the first stent of FIG. 5A showing growth of the tissues from outer layers of the esophagus within a hollow region or lumen of the first stent, in accordance with an embodiment of the present specification; and

FIG. 5C is a diagrammatic representation of a second stent positioned within the hollow region or lumen of the first stent of FIG. 5B, such that a length of the second stent substantially overlaps a length of the first stent, in accordance with an embodiment of the present specification.

### DETAILED DESCRIPTION

The present specification discloses an implantable device and method for the treatment of Barrett's esophagus, a premalignant condition. Barrett's esophagus is a serious complication of GERD. It is characterized by the replacement of the normal stratified squamous epithelium lining of the esophagus by simple columnar epithelium with goblet cells (which are usually found lower in the gastrointestinal tract). In an embodiment, the present specification describes an implantable device that is deployed to cause necrosis of the mucosal layer of the Barrett's esophagus through induction pressure. In an embodiment, the present specification describes a method comprising deploying multiple stents to cause pressure ischemia and subsequent necrosis of a mucosal layer of the esophagus that is affected with Barrett's syndrome.

In an embodiment, the present specification describes performing a radio frequency ablation (RFA) treatment on an esophageal lesion, which is affected with the Barrett's disease. Subsequently, a first completely uncovered or partially covered self-expanding stent is deployed in the esophagus such that the first stent covers the area of the esophageal lesion, which is affected with the Barrett's disease. In embodiments, the term “cover” or “covered” means that a material, such as silicone, polyethylene, or polyurethane, is applied over an underlying porous substrate, such as the wire mesh stent structures described herein, thereby creating a region through which fluid and tissue will not flow and substantially eliminating the porosity of the underlying porous substrate. It should be appreciated that a covered region is differentiated from a non-covered region in that the covered region has material that overlays the underlying substrate (e.g. wire mesh) while the non-covered region has the holes of the wire mesh substantially exposed to the underlying (esophageal) tissue.

After a period of a few days, or once the first stent expands and embeds itself within the esophagus, a second fully or partially covered self-expanding stent is deployed such that the second stent is positioned within the same hollow region or lumen as the first stent, and within a lumen of the first stent, and such that a length of second stent substantially overlaps a length of the first stent. The application of the second stent causes pressure ischemia and subsequent necrosis of the affected mucosal layer of the esophagus that is sandwiched between the two stents.

In various embodiments, if a stent that is positioned outside the area of the Barrett's esophagus is partially covered, it may be easier to remove. In an embodiment, only a proximal and/or distal part of the first stent is covered. In embodiments, a proximal flange portion and a distal flange portion of the first stent are covered. For example, a length of the covered portion may range from 0.5 cm to 4 cm. In an embodiment, only the uncovered portion of the first stent is embedded within the affected region of the Barrett's esophagus while the covered portion of the stent is placed outside the affected area of Barrett's esophagus. In embodiments, the portion of the first stent covering the affected area (esophageal lesion or Barrett's tissue) and extending a maximum of 2 cm above and 2 cm below the affected area is uncovered. In an embodiment, the second stent is partially covered, with a proximal and/or distal part of the second stent being uncovered, where the length of the uncovered portion may range from 0.5 cm to 4 cm. In various embodiments, the covered portions of the first and the second stent may be covered with materials such as, but not limited to, silicone, polyethylene and polyurethane.

After a few days, the two stents are removed and the affected area is reexamined. Optionally, RF ablation is performed again on the treated region to ensure that the affected tissues are completely removed. A complete necrosis of the affected layer of the esophagus leads to eradication of the Barrett's disease.

As the second stent is fully or partially covered, it may not have sufficient anchoring support and is therefore prone to migration within the esophagus and/or lumen of the first stent. In embodiments, the methods and systems disclosed are designed to ensure that the second stent does not migrate into the esophagus. In some embodiments, the second stent is coupled to the first stent through at least one coupling mechanism, such as a magnet, hooks or glue.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

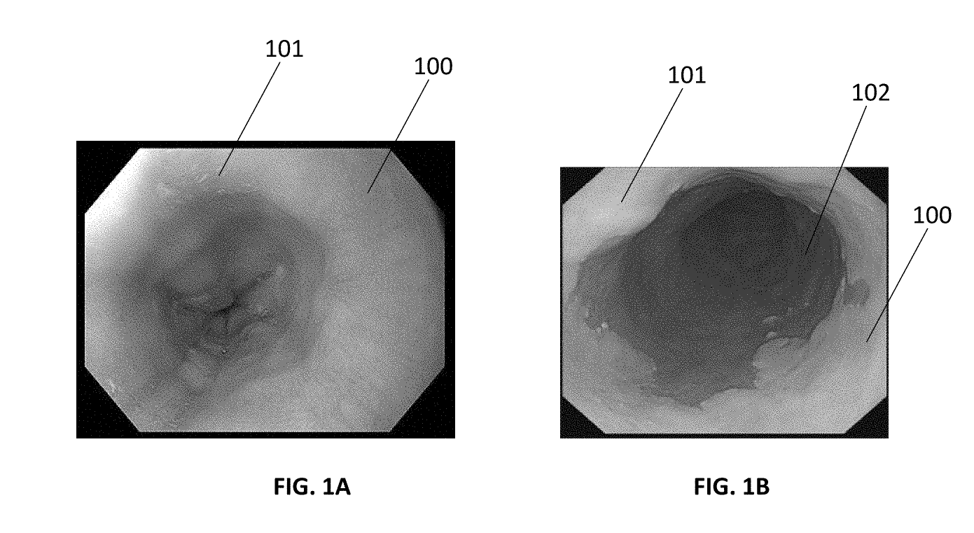

FIG. 1A illustrates an epithelium lining of a normal healthy esophagus. As shown in FIG. 1A, the epithelium lining 101 of the esophagus 100 is in a healthy condition and does not have any abnormality. FIG. 1B illustrates an epithelium lining in an esophagus affected with Barrett's syndrome. In patients with frequent acid reflux, the normal cells within the esophagus are replaced by cells that are similar to cells in the intestine and may eventually lead to a Barrett's esophagus condition. As shown in FIG. 1B, certain portions of the esophagus 100 are covered with a lining 102, which resembles a tissue lining generally found in the intestine. The areas which have a tissue lining such as the lining 102 are affected with the Barrett's syndrome.

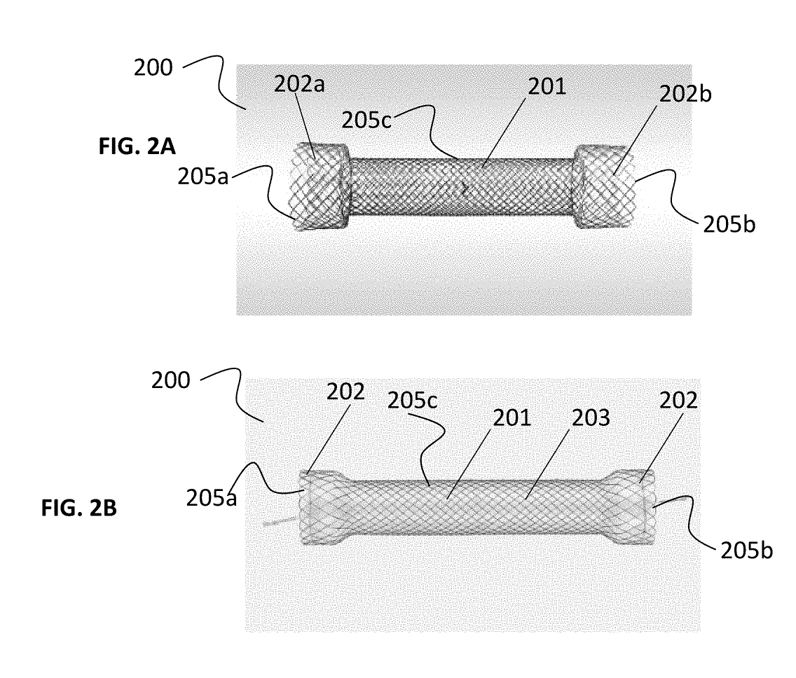

FIG. 2A illustrates an exemplary embodiment of a partially uncovered stent which may be deployed in accordance with an embodiment of the present specification. As shown in FIG. 2A, the stent 200 comprises a hollow cylindrical tube 201 which is used in order to hold open a structure or lumen within a gastrointestinal tract. In embodiments, the lumen of the gastrointestinal tract is the esophagus. In embodiments, the body or hollow cylindrical tube 201 of the stent comprises a metal mesh, thus, as the stent 200 is firmly positioned, the tissues of the outer layers of the esophagus start growing towards the inner surface of the stent through the metal mesh. In an embodiment, the stent 200 is manufactured using an alloy such as Nitinol. In an embodiment, one or more anchoring magnets may be positioned on or within a proximal end 205a, a distal end 205b and a center or middle portion 205c of stent 200. In embodiments, the positions of the anchoring magnets may vary along the length of stent 200. In embodiments, the portion of the first stent covering the affected area (esophageal lesion or Barrett's tissue) and extending a maximum of 2 cm above and 2 cm below the affected area is uncovered. The stent 200 further comprises flanges 202a, 202b which allow for anchoring of the stent within the esophagus to prevent migration. In embodiments, proximal flange 202a and distal flange 202b of the first stent are covered. In embodiments, the length of each flange ranges from 0.5 cm to 6 cm.

FIG. 2B illustrates an exemplary embodiment of a partially uncovered stent which can be deployed in accordance with an embodiment of the present specification. In alternate embodiments, stent 200 may be fully covered. As shown in FIG. 2B, the stent 200 comprises a hollow cylindrical tube 201 comprising a metal mesh which is covered with a fiber or coating to prevent or reduce in-growth of the tissues from outer layers of the esophagus in the stent 200. In embodiments, the fiber is a silicon fiber. In embodiments, the cover or coating is silicone, polyethylene or polyurethane. Since the migration rates are very high in a fully covered stent, the present embodiment describes using various coupling mechanisms between the first stent and the second stent. In an embodiment, one or more anchoring magnets may be positioned on or within a proximal end 205a, a distal end 205b and a center or middle portion 205c of stent 200. In embodiments, the positions of the anchoring magnets may vary along the length of stent 200. In embodiments, the stent 200 further comprises flanges 202 which allow anchoring the stent in the esophagus. In various embodiments, flanges 202 are provided on at least a proximal end 205a of stent 200 and may be cup-shaped or tulip-shaped. In an embodiment, flanges 202 may also be provided on a distal end 205b of stent 200. In embodiments, the proximal flange portion and the distal flange portion of the second stent are uncovered. Optionally, the uncovered flange portions serve to anchor the second stent within the lumen of the esophagus. In embodiments, the length of each flange ranges from 0.5 cm to 6 cm.

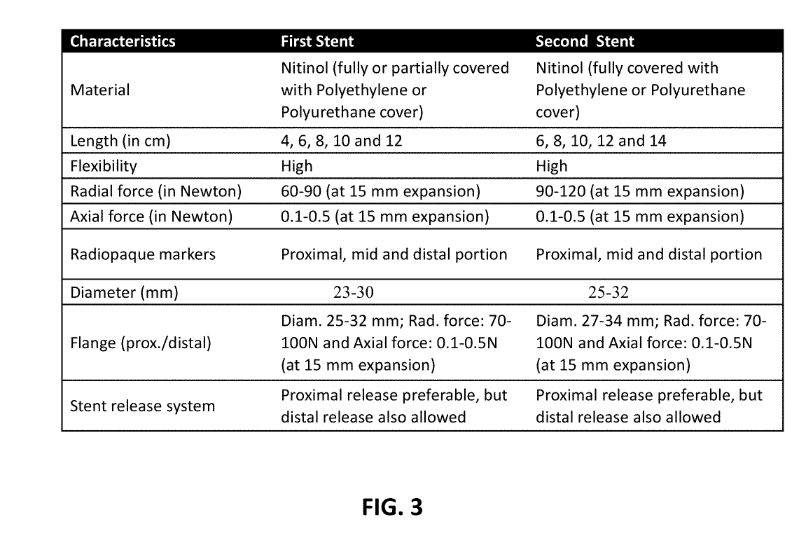

FIG. 3 is a table depicting exemplary properties of the two stents in accordance with an embodiment of the present specification. As shown in the table, in an embodiment, a first stent and a second stent are manufactured using an alloy such as Nitinol. In an embodiment, the first stent is fully or partially uncovered and the second stent is fully or partially covered with silicone, polyethylene or polyurethane material. In an embodiment, the first stent is manufactured in a variety of configurations having a range of lengths, including but not limited to 4 cm, 6 cm, 8 cm, 10 cm and 12 cm. The second stent is also available in a variety of configurations having a range of lengths, including but not limited to 6 cm, 8 cm, 10 cm, 12 cm and 14 cm. It should be noted that in embodiments of the present specification, for a specific procedure, the length of the second stent is typically approximately 2-4 cm greater than the length of the first stent. In embodiments, the length of each flange portion ranges from 0.5 cm to 6 cm.

In embodiments, both the first stent and the second stent comprise a high level of flexibility. At approximately 15 mm expansion, the first stent has a radial force between 60-90 Newton and the second stent provides a radial force between 90-120 Newton. Further, at 15 mm expansion, both the first stent and the second stent provide an axial force between 0.1-0.5 Newton. In an embodiment, the diameter of the first stent is 20-32 mm and the diameter of the second stent varies between 22-34 mm. In an embodiment, the first stent comprises a flange having a diameter between 24-36 mm, a radial force between 70-100N and an axial force between 0.1-0.5N (at 15 mm expansion). In an embodiment, the second stent comprises a flange having a diameter between 26-38 mm, a radial force between 70-100N and an axial force between 0.1-0.5N (at 15 mm expansion). In embodiments, the first stent and the second stent comprise a proximal release system. In another embodiment, the first stent and the second stent comprise a distal release system. Stents may be released through any mechanism, including detaching the magnetic elements, removing the physical hooks, anchors, or other members, and/or cutting the wires or sutures, which keep the stents in place.

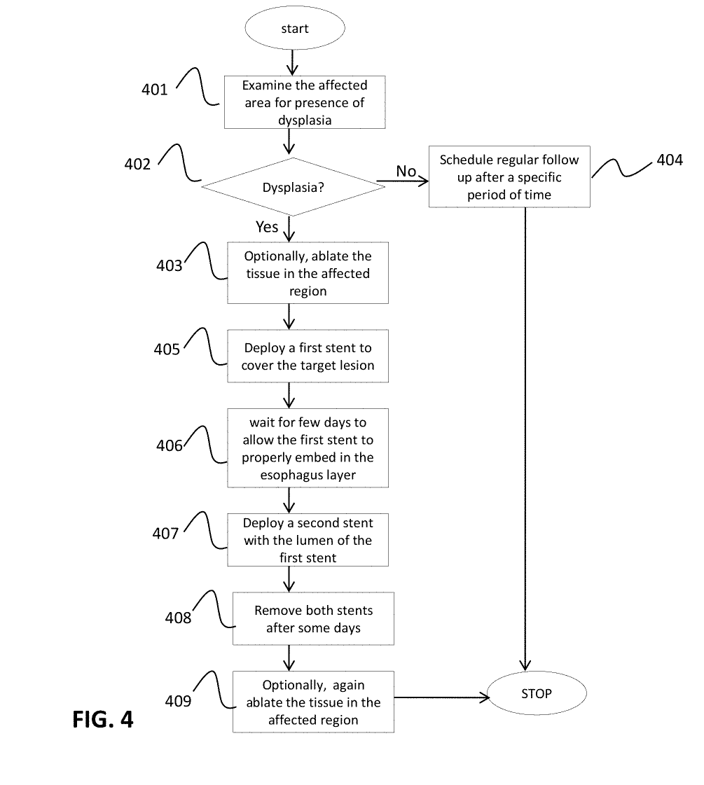

FIG. 4 is a flow chart depicting a plurality of steps for treating Barrett's esophagus in accordance with an embodiment of the present specification. As shown in the flow chart in FIG. 4, at step 401, a patient having Barrett's esophagus with a confirmed diagnosis of dysplasia is examined to determine if he/she has signs of dysplasia in the esophagus. In an embodiment, an esophagus affected with Barrett's syndrome may appear as shown in FIG. 1B. Dysplasia refers to the proliferation of cells of an abnormal type, as a developmental disorder or an early stage in the development of cancer. Usually, in cases where a Barrett's esophagus condition progresses towards a cancerous development, it is visible in the form of dysplasia. At step 402, if the examination does not show any signs or symptoms of dysplasia, the patient is advised to schedule a routine check-up at step 404 to periodically monitor the status of his Barrett's esophagus condition. Conversely, if the examination shows the presence of dysplasia, at step 403, depending on the condition of the dysplasia, optionally, the affected region is treated through RF ablation. In RF ablation, radiofrequency energy (radio waves) is delivered via a catheter to the esophagus to remove or “ablate” a diseased tissue while minimizing injury to healthy esophagus tissue.

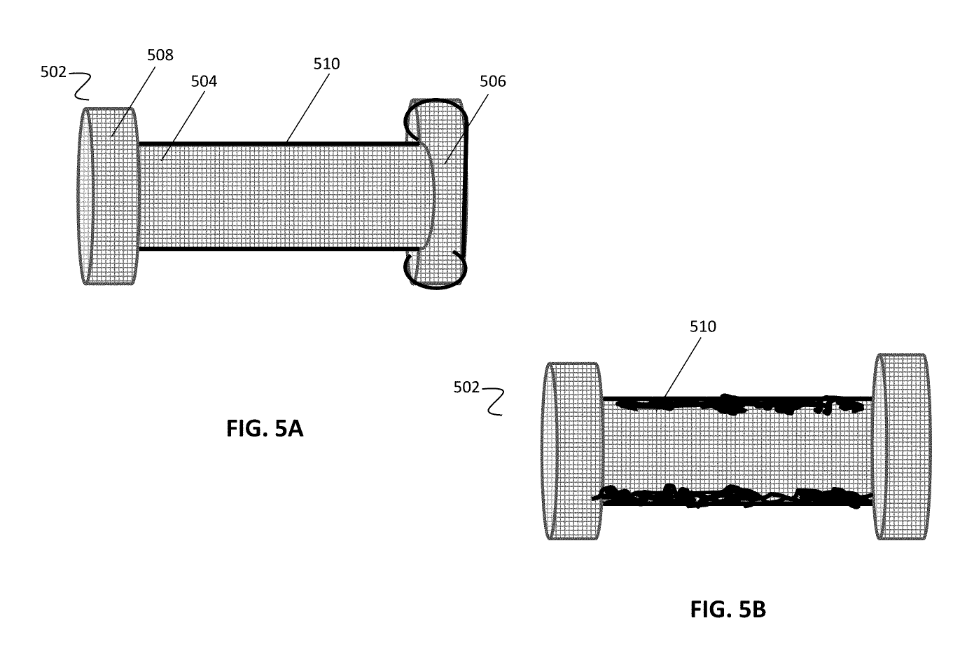

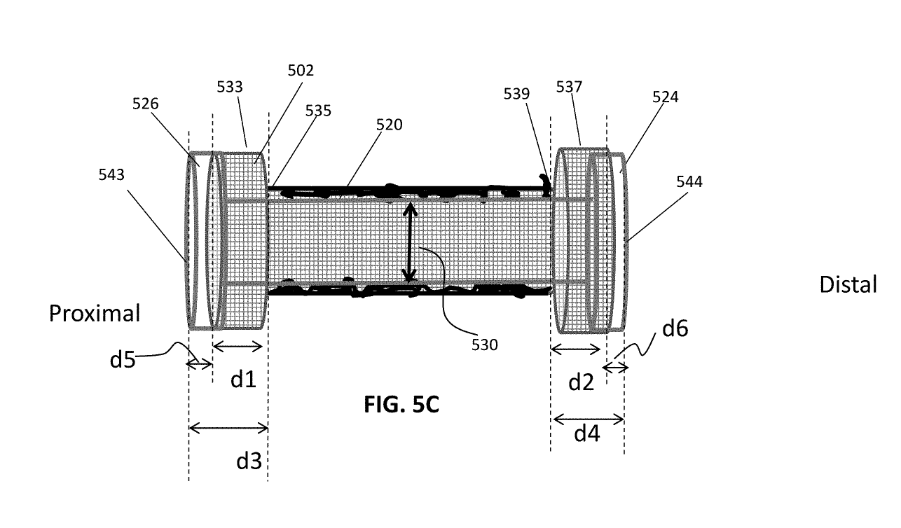

Subsequently, post RF ablation (if performed), a first stent is deployed such that an outer surface of the first stent is in physical communication with the layers of tissue within the esophagus which are affected with the Barrett's syndrome. In embodiments, the first stent has a hollow cylindrical shape that comprises a metal or non-metal mesh. FIG. 5A is a diagrammatic representation of a first stent deployed in Barrett's esophagus with a confirmed diagnosis of dysplasia, in accordance with an embodiment of the present specification. First stent 502 is made of a metallic or non-metallic mesh material such as shown in FIG. 2A, and comprises a hollow cylindrical body 504 with flanges 506 and 508 provided at a distal and a proximal end, respectively. Outer layers of esophageal tissue 510 surround the stent 502 when the stent is in a deployed position. In embodiments, the first stent is selected such that it at least fully covers an esophageal lesion or affected area. In an embodiment, the first stent 502 is substantially uncovered i.e. a large portion of the stent does not have any fiber cover over the metal mesh. In embodiments, the portion of the first stent covering the affected area (esophageal lesion or Barrett's tissue) and extending a maximum of 2 cm above and 2 cm below the affected area is uncovered. In embodiments, a covered portion of the first stent ranges from 0.5 cm to 4 cm. Substantially uncovered stents have at least a portion of exposed bare metal at the proximal and distal ends to allow the stent to embed into the esophageal wall, which helps to prevent migration of the stent from its deployed position. Absence of the fiber cover allows the growth of the tissues from the outer layers of the esophagus to inside the hollow region of the stent. In an embodiment, the first stent comprises a self-expandable metallic or non-metallic stent (or SEMS) which is a metallic or non-metallic tube, or stent, used in order to hold open a structure in the gastrointestinal tract. In embodiments, the patient is discharged after the first stent is deployed and is required to return after few days for a second procedure. During the course of time (usually a few days or a week), due to its self-expanding property, the first stent is positioned in the esophagus such that the portions of the stent embed within the outer layers of the esophagus tissue resulting in growth of the tissue towards an inside region or inner lumen of the stent.

As shown in step 406, there is a first pre-determined waiting period of 7-10 days, during which the tissues from the outer layers of the esophagus grow within the first stent. In embodiments, the first pre-determined waiting period varies for different patients and may range between 5 to 14 days. FIG. 5B is a diagrammatic representation of the first stent 502 of FIG. 5A with growth of the tissues from outer layers 510 of the esophagus towards an inside region or inner lumen of the first stent, in accordance with an embodiment of the present specification. As shown, tissue 510 grows and embeds into stent 502 through the mesh body of the stent.

At step 407, a second procedure is conducted in which a second stent is deployed within the esophagus such that it is positioned within the same hollow region of the first stent and such that the second stent substantially overlaps (lengthwise) and is positioned with the lumen of the first stent. In an embodiment, the length of the second stent covers at least the entire length of the first stent. In an embodiment, a second pre-determined waiting period ranges from 5-14 days, and preferably 7-10 days such that if the first procedure is conducted at t=0 day, the second procedure is conducted at t=7-10 days.

FIG. 5C is a diagrammatic representation of a second stent positioned within the hollow region or lumen of the first stent of FIG. 5B, such that a length of the second stent substantially overlaps a length of the first stent, in accordance with an embodiment of the present specification. A second stent 520 comprises a hollow cylindrical body with flanges 524 and 526 provided at a distal end and a proximal end respectively, and is placed within the lumen of the first stent 502. In an embodiment, the second stent 520 has a hollow cylindrical shape that comprises a metallic or non-metallic mesh. In an embodiment, the mesh is fully or substantially covered with a fiber such that the presence of fiber does not allow the tissues from the outer layers of esophagus to grow into the hollow region of the second stent. In an embodiment, the first stent sheaths the second stent, which causes the affected layers of esophagus to be “sandwiched” between the first stent and the second stent.

In an embodiment, a radial force 530 of the second stent on the well-vascularized Barrett's tissue of the first stent causes a pressure ischemia and subsequent necrosis of the tissue layers present between the two stents. The second stent remains in its position for the second pre-determined waiting period of a few days and subsequently, at step 408, a third procedure is conducted in which both the first stent and second stent are removed. In an embodiment, the two stents remain in a deployed position for a total time period ranging between 10 and 14 days after the second procedure which means if the second procedure is conducted at t=7 days, the third procedure is conducted at t=14-17 days (ranging from 12-21 days). In an embodiment, the first stent and the second stent are removed using forceps such as rat-tooth grasping forceps, or a snare. At step 409, optionally, RF ablation is again performed on the treated region to ensure that affected tissues are completely removed. In an embodiment, a normal healthy esophagus after removal of affected tissue appears as shown in FIG. 1A.

Subsequently, follow-up endoscopies are recommended after a period of 14 days (total time t=26-35 days) and 90 days (total time t=116-125 days) to evaluate the eradication of Barrett's esophagus. In an embodiment, a first follow up endoscopy is conducted after 14 days, a second follow up endoscopy after 45 days and a third follow up endoscopy after 90 days of the removal of two the stents to evaluate the eradication of Barrett's esophagus. If Barrett's esophagus is not eradicated successfully, steps 402 to 409 depicted in the flowchart in FIG. 4 are repeated.

In an embodiment, the first stent and the second stent are positioned via an endoscopic procedure. In another embodiment, the first stent and the second stent are positioned using fluoroscopy.

In an optional embodiment, after the placement of a first stent the patient is prescribed a medication. In an embodiment, the patient is prescribed 20 mg Omeprazole BID dose for the full treatment period (i.e. t=0-(121-125) days). In addition, the patient may also be prescribed sucralfate 10 mg, 4 times a day, from start of the procedure until the date of first follow up endoscopic procedure (i.e. t=0-(31-35) days). In an embodiment the patient is administered a prescription medicine Omeprazole 40 mg BID dose or equivalent dose of an alternative PPI for the full treatment period from start of the procedure until the date of third follow up endoscopic procedure. In another embodiment, the patient is administered a prescription medicine sucralfate 10 mg, 4 times from start of the procedure until the date of first follow up endoscopic procedure. In another embodiment, the patient is administered a prescription medicine Ranitidine 300 mg, once at night time from start of the procedure until the date of first follow up endoscopic procedure.

Referring back to FIG. 5C, in an embodiment, the proximal end 533 of the first stent 502 is placed at a distance ‘d1’ ranging from 1 cm to 2 cm beyond a first end 535 of an esophageal lesion to be treated and the distal end 537 of the first stent 502 is placed at a distance ‘d2’ ranging from 1 cm to 2 cm beyond a second end 539 of the esophageal lesion to be treated. Thus, the total length of the first stent 502 is approximately 2 cm to 4 cm longer (2×(1 cm to 2 cm) on each of the distal and proximal ends) than the length of the esophageal lesion. In an embodiment, the minimum and maximum lengths of the first stent 502 are 4 cm and 12 cm respectively.

In an embodiment, the proximal end 543 of the second stent 520 is placed at a distance d3′ ranging from 2 cm to 4 cm beyond a first end 535 of the esophageal lesion to be treated and the distal end 544 of the second stent 520 is placed at a distance ‘d4’ ranging from 2 cm to 4 cm beyond a second end 539 of the esophageal lesion to be treated. Stated differently, in an embodiment, the proximal end 543 of the second stent 520 is placed at a distance ‘d5’ of no more than 2 cm extending from the proximal end 533 of the first stent 502 and the distal end 544 of the second stent 520 is placed at a distance ‘d6’ of no more than 2 cm extending from the distal end 537 of the first stent 502. Thus, the total length of the second stent 520 is approximately 2 cm to 4 cm longer (1 cm to 2 cm on each of the distal and proximal ends) than the length of the first stent 502. In a preferred embodiment, a length of the first stent 502 is at least 2 cm greater than at least a length of the affected region and a length of the second stent 520 is at least 2 cm greater than a length of the first stent 502.

In an embodiment, the second stent is fully covered and does not have any exposed bare metal to anchor itself along the esophageal wall and is therefore prone to migration in the esophagus tract. In an embodiment, the second stent is partially uncovered, with a proximal and/or distal part of the second stent being uncovered, and the length of the uncovered portion ranging from 0.5 cm to 4 cm. In embodiments, the proximal flange portion and the distal flange portion of the second stent are uncovered. Optionally, the uncovered flange portions serve to anchor the second stent within the lumen of the esophagus. In various embodiments, the covered portions of the first and the second stent may be covered with materials such as, but not limited to, silicone, polyethylene and polyurethane.

The present specification describes systems and methods to prevent migration of the second stent. In an embodiment, the first stent and the second stent comprise magnetic materials such that the resulting magnetic field secures the second stent in a firm position and prevents its migration. In another embodiment, adhesives such as tissue glue (e.g. histoacryl) or fibrin glue (e.g. tissucol) is deployed on the polytetrafluoroethylene (PTFE) based cover of the second stent which couples the second stent with the surrounding tissues. In an embodiment, the second stent is configured such that the adhesive is released only after placement of the stent in its position. In an embodiment, the first stent and the second stent comprise radiopaque markers at proximal, mid and distal portions to monitor positioning.

In an embodiment, the first stent is heated to a maximum temperature of 30 to 70 degrees Celsius, preferably about 53 degrees Celsius, for causing additional necrosis of the Barret's tissue. In another embodiment, the system is configured such that monopolar or bipolar energy is delivered to the first stent. In another embodiment, the system is configured to deliver radio frequency ablation to the first stent.

In an embodiment, the profile of a suitable patient candidate for the above procedures is defined as follow:
The patient should have Barrett's esophagus, defined as columnar epithelium with or without histologically proven intestinal metaplasia
The length of the Barrett's esophagus should be between 2 cm and 10 cm
There should not be any endoscopically visible lesions in Barrett's esophagus
Age of the patient should be over twenty one years
Patient should not be pregnant
Patient should not be on anticoagulants or have other bleeding disorder.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for treating Barrett's esophagus in a patient comprising:
identifying an affected region in the esophagus;
deploying an at least partially uncovered first stent within the esophagus wherein the first stent overlaps at least the entire affected region in the esophagus;
leaving said first stent within the esophagus for a first predetermined time period;
deploying an at least partially covered second stent within the esophagus after said predetermined time period such that the second stent is positioned within the first stent, thereby positioning the first stent between the second stent and the affected region in the esophagus, and extends beyond the first stent by a predefined distance; and,
removing the first stent and second stent after a second predetermined time period.

2. The method of claim 1, further comprising ablating tissues in the affected region before deploying the first stent.

3. The method of claim 1, further comprising ablating tissues in the affected region after removing the first stent and the second stent.

4. The method of claim 1, wherein said first predetermined time period is at least seven days from the deploying of the first stent.

5. The method of claim 1, wherein said second predetermined time period ranges from ten to fourteen days from the deployment of the second stent.

6. The method of claim 1, wherein the second stent is coupled to the first stent to prevent migration within the esophagus.

7. The method of claim 6, wherein the second stent is coupled to the first stent using a magnetic field.

8. The method of claim 6, wherein the second stent is coupled to the first stent using at least one of a mechanical member and an adhesive.

9. The method of claim 1, wherein when said second stent is positioned within said first stent, a mucosal layer of the esophagus is sandwiched between the first and second stents under an amount of pressure sufficient to cause pressure ischemia and subsequent necrosis of the mucosal layer of the esophagus.

10. The method of claim 1, wherein a diameter of the first stent ranges between 20 and 32 mm and a diameter of the second stent ranges between 22 and 34 mm.

11. The method of claim 1, wherein a length of the first stent is at least 2 cm greater than at least a length of the affected region and a length of the second stent is at least 2 cm greater than a length of the first stent.

12. The method of claim 1, wherein a radial force of the first stent ranges between 60 and 90 Newton at approximately 15 mm expansion and a radial force of the second stent ranges between 90 and 120 Newton at approximately 15 mm expansion.

13. The method of claim 1, wherein an axial force of the first stent ranges between 0.1 and 0.5 Newton at approximately 15 mm expansion and an axial force of the second stent ranges between 0.1 and 0.5 Newton at approximately 15 mm expansion.

14. The method of claim 1, wherein the first stent and the second stent comprise radiopaque markers positioned on at least one of a proximal end, center portion and distal end.

15. The method of claim 1, wherein the first stent comprises a flange having a diameter ranging between 24 and 36 mm, and a length ranging from 0.5 cm to 6 cm and wherein the first stent provides a radial force ranging between 70 and 100 Newton and an axial force ranging between 0.1 and 0.5 Newton at 15 mm expansion.

16. The method of claim 1, wherein the second stent comprises a flange having a diameter ranging between 26 and 38 mm, and a length ranging from 0.5 cm to 6 cm and wherein the second stent provides a radial force ranging between 70 and 100 Newton and an axial force ranging between 0.1 and 0.5 Newton at 15 mm expansion.

17. The method of claim 1, wherein at least one of a proximal and a distal portion ranging in length from 0.5 to 4 cm of the first stent is partially covered and wherein the covered portion of the first stent is placed outside the affected region and wherein at least one of a proximal and distal portion ranging in length from 0.5 to 4 cm of the second stent is partially uncovered.

18. The method of claim 17 wherein covered portions of the first stent and second stent comprise material made of at least one of silicone, polyethylene, and polyurethane.

19. The method of claim 1, wherein the first stent is configured such that it can be released through at least one of a proximal section of the first stent and a distal section of the first stent.

20. The method of claim 1, wherein the second stent is configured such that it can be released through at least one of a proximal section of the second stent and a distal section of the second stent.
